(19) United States
(12) Patent Application Publication
(10) Pub. No.: US 2012/0253440 A1
(43) Pub. Date: Oct. 4, 2012

(54) DEVICE AND METHOD FOR ENSURING THE PROPER INSERTION OF A LEAD INTO THE HEADER OF AN IMPLANTABLE MEDICAL DEVICE

(75) Inventor: Elizabeth Grohmann, Frisco, TX (US)

(73) Assignee: ADVANCED NEUROMODULATION SYSTEMS, INC., Plano, TX (US)

(21) Appl. No.: 13/237,521

(22) Filed: Sep. 20, 2011

Related U.S. Application Data
(60) Provisional application No. 61/428,558, filed on Dec. 30, 2010.

(51) Int. Cl. A61N 1/05; H01R 3/00

(52) U.S. Cl. 607/116; 439/488

(57) ABSTRACT

An implantable medical device that includes a header a cavity or connector bore that extends from an opening at a first end of the header towards a second end of the header. The cavity is configured to receive a first end of a stimulation lead. The header includes a lead insertion indicator positioned at the second end of the cavity for engaging with the stimulation upon complete insertion into the header. The lead insertion indicator is operable between a first operating state and a second operating state, such that when the stimulation lead is not engaged with the lead insertion indicator, the lead insertion indicator operates in the first operating state, and when the stimulation lead properly engages the lead insertion indicator, the lead insertion indicator operates in the second operating state. The lead insertion indicator generates a signal that indicates the corresponding operating state.

### TECHNICAL FIELD

The present invention relates to medical devices having leads associated therewith, and more particularly, but not by way of limitation, to a device and method for providing verification of proper lead insertion into either the header or connector of an implantable medical device.

### BACKGROUND

Numerous medical devices have been developed and used from providing various types of therapy to patients. Some of the medical devices utilize electrical leads for delivering therapy and/or monitoring various physiological conditions of the patient. The electrical lead carries sensors or electrodes for deployment to the targeted therapy delivery or monitoring site. For example, implantable electrical leads are commonly used to form part of implantable neurostimulation systems in which a pulse generator and electrical leads are implanted in a patient to provide pain management through controlled electro-stimulation via electrodes disposed on the electrical leads, typically near the distal ends of the leads.

The electrodes are coupled to conductors extending to the proximal end of the electrical lead where each conductor is coupled to a connector included in a lead connector assembly. The pulse generator is generally provided with a connector header having cavities adapted for receiving a corresponding lead connector assembly. The connector cavities have electrical contacts which mate with the connectors included on the lead connector assembly. When the lead connector assembly is properly inserted in the connector cavity, the electrodes carried by the electrical lead are electrically coupled to the circuitry contained in the pulse generator via feedthroughs which connect the connector header contacts to the circuitry contained in the pulse generator. Thus, proper insertion of the lead connector assembly into the connector cavity is essential for proper operation and therapy delivery of the of neurostimulation system.

Some implantable device systems utilize a visual lead insertion indicator for the verification of proper electrical lead insertion. For example, some implantable device systems utilize a visibly modified surface on the electrical lead to facilitate visual verification of full insertion into the connector cavity of the header. A disadvantage of the visual indicator is that it provides no benefit once the device has been implanted into the patient. Because the visual indicators can no longer be seen after being implanted, if a lead pulls at least partially out of the connector cavity, an x-ray or explant would be needed to verify this.

It would be advantageous to have a system and method for ensuring and verifying the proper and continuous insertion of a lead into the header of an implantable medical device that overcomes the disadvantages of the prior art. The present invention provides such a system and method.

### BRIEF DESCRIPTION OF THE SEVERAL VIEWS OF THE DRAWING

In the following section, the invention will be described with reference to exemplary embodiments illustrated in the figures, in which:

FIG. 1 illustrates one embodiment of an implantable stimulation system which may utilize certain aspects of the present invention;

FIG. 2 is block diagram of a stimulation system which may utilize certain aspects of the present invention;

FIG. 3 is a cross-sectional view of a header as similarly shown in FIG. 1;

FIG. 4 illustrates a portion of the cavity of a header and lead insert indicator of a header for receiving a lead which may utilize certain aspects of the present invention;

FIG. 5 illustrates the portion of the cavity illustrated in FIG. 4 with a lead inserted therein and engaging the lead insert indicator;

FIG. 6 illustrates a dual lead insert indicator which may be utilized in certain configurations of headers for receiving two leads;

FIG. 7 illustrates another embodiment of a portion of cavity of a header and lead insert indicator of the header for receiving a lead which may utilize certain aspects of the present invention; and

FIG. 8 illustrates the a portion of a cavity as similarly illustrated in FIG. 7 with a lead inserted and engaging the lead insert indicator.

### DETAILED DESCRIPTION

For the purposes of promoting an understanding of the principles of the present invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

When directions, such as upper, lower, top, bottom, clockwise, counter-clockwise, are discussed in this disclosure, such directions are meant to only supply reference directions for the illustrated figures and for orientations of components in the figures. The directions should not be read to imply actual directions used in any resulting invention or actual use. Under no circumstances, should such directions be read to limit or impart any meaning into the claims.

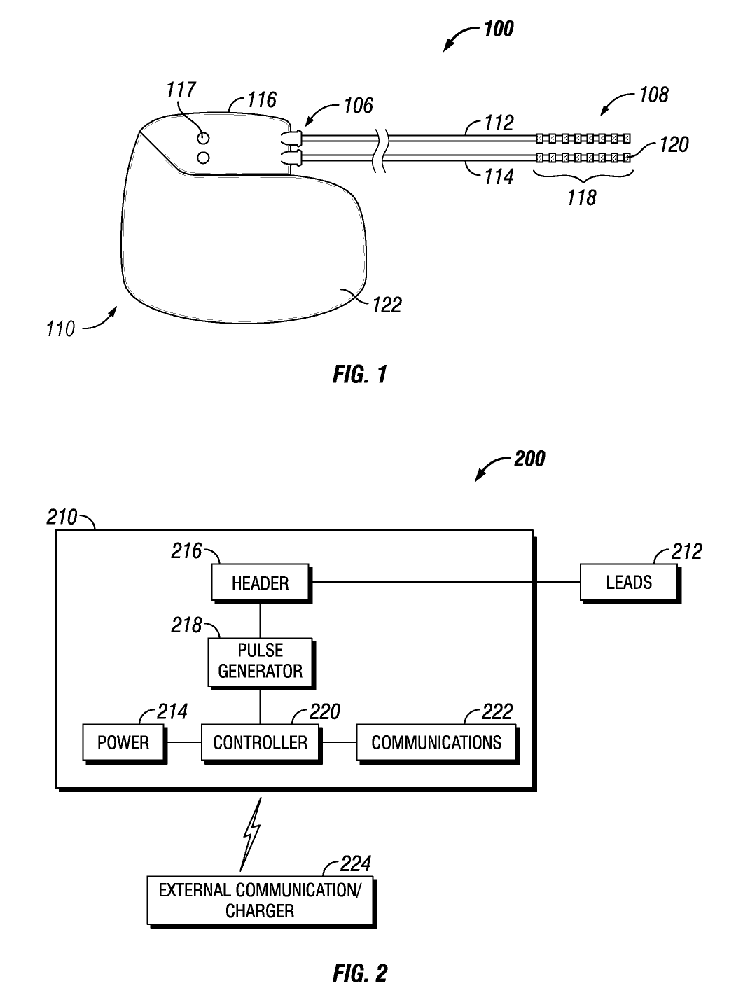

Referring now to FIG. 1, there is illustrated an exemplary implantable stimulation system 100 which may employ certain aspects of the present invention. Stimulation system 100 includes a stimulation source, such as an implantable pulse generator 110 (“IPG”) which may be coupled to one or more stimulation leads 112 and 114. As will be explained below, the IPG 110 typically includes a power source (such as a battery) and electronics (such as hardware, software, or embedded logic components) for generating electrical stimulation signals or pulses.

In this example, the stimulation system 100 employs the two stimulation leads 112 and 114 inserted into and connected to the header 116 of IPG 110, but any number of stimulation leads could be employed and are within the scope of the present invention. Each of the leads 112 and 114 may generally be configured to transmit one or more electrical signals from the IPG 110 to a spinal nerve, a peripheral nerve, or other tissue. As illustrated in FIG. 1 by the break lines, the leads 112 and 114 are not meant to accurately represent the actual length of the leads relative to the pulse generator. Header 116 utilizes set-screws 117 to engage and mechanically secure leads 112 and 114 to substantially inhibit the removal of leads 112 and 114 from header 116 (see FIGS. 4 and 5).

Each of the leads 112 and 114 include a proximal end 106 and a distal end 108 and comprises a flexible lead body that extends from proximal end 106 to the distal end 108. In certain embodiments, one or more lumens (not shown) may extend through leads 112 and 114 and may be used for housing one or more stiffeners (not shown).

In certain embodiments, each of the leads 112 and 114 may be a structure having a round or substantially round cross-section. Alternatively, the cross-section of each of the leads 112 and 114 may be configured in any number of cross-sectional shapes appropriate for a specific application in which the lead will be used. Depending on the particular application, the diameter of the lead may be any size, though a smaller size is more desirable for lead applications such as neurological and myocardial mapping/ablation and neuromodulation and stimulation.

The leads 112 and 114 may each be formed of an extrusion or insulating material typically selected based upon biocompatibility, biostability and durability for the particular application. The insulator material may be silicone, polyurethane, polyethylene, polyamide, polyvinylchloride, PTFT, EFTE, or other suitable materials known to those skilled in the art. Alloys or blends of these materials may also be formulated to help control the relative flexibility, torqueability, and pushability of the lead. In some applications, compliant material characteristic enables the each of the leads 112 and 114 to elongate significant amounts at relatively low stretching forces.

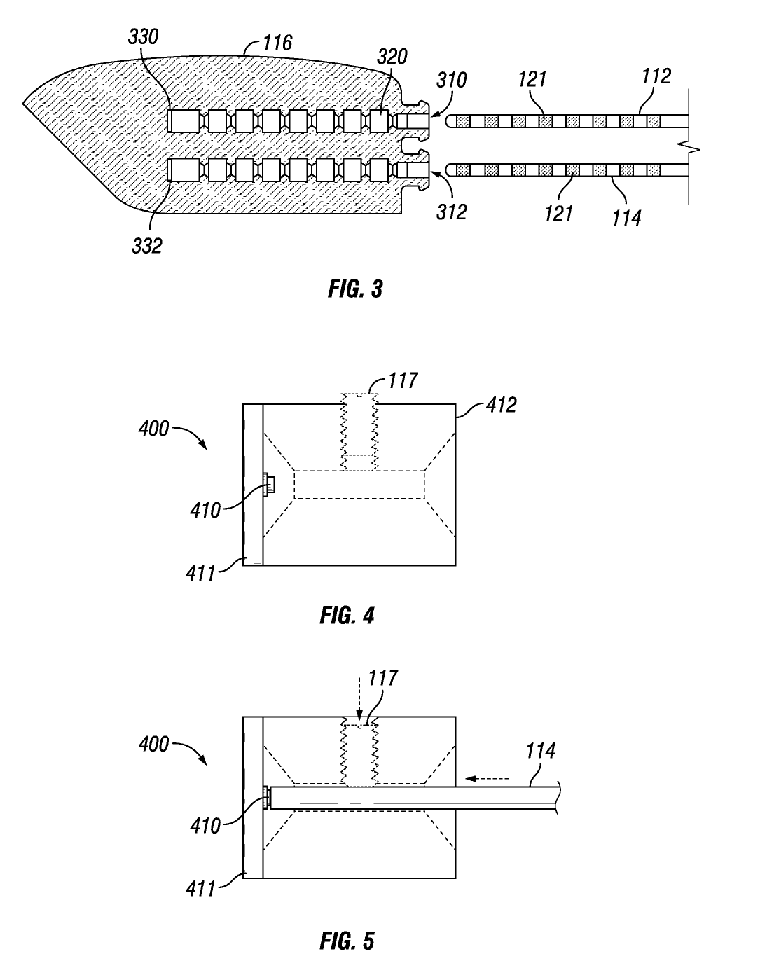

Adjacent to the distal end 108 of each of the leads 112 and 114 is a stimulation electrode region 118 comprising, in this embodiment, a plurality of eight stimulation electrodes 120 on each of the leads 112 and 114. Each of the leads 112 and 114, at proximal end 106, comprises a corresponding plurality of eight connector or terminal electrodes 121 sized to couple with the header 116 (See FIG. 3). As will be appreciated by those skilled in the art, any number of corresponding conductors and electrodes may be utilized at the two ends of leads 112 and 114.

In certain embodiments, the electrodes may be formed of biocompatible, conductive materials such as stainless steel, platinum, gold, silver, platinum-iridium, stainless steel, MS35N, or other conductive materials, metals or alloys known to those skilled in the art. In some embodiments, the electrodes may be ring or cylindrical electrodes which encircle ends 106 and 108 of leads 112 and 114. Other types, configurations and shapes of electrodes as known to those skilled in the art may be used with embodiments.

One or more conductors (not shown) extend along a substantial portion of each of the leads 112 and 114 to electrically connect the corresponding electrodes between ends 106 and 108. The one or more conductors of lead 112 and 114 may be maintained in electrical isolation by insulative material of each of the leads 112 and 114.

In certain embodiments, the conductors may be formed of a conductive material having desirable characteristics such as biocompatibility, corrosion resistance, flexibility, strength, low resistance, etc. The conductors may take the form of solid wires, drawn-filled-tube (DFT), drawn-brazed-strand (DBS), stranded wires or cables, ribbon conductors, or other forms known or recognized to those skilled in the art. The composition of the conductors may include aluminum, stainless steel, MP35N, platinum, gold, silver, copper, vanadium, alloys, or other conductive materials or metals known to those of ordinary skill in the art. In some embodiments, the number, size, and composition of the conductors will depend on the particular application for the lead, as well as the number of electrodes.

The IPG 110 includes a housing 122 to enclose elements (described herein below) for generating the electrical pulses for application to neural tissue of the patient. The elements enclosed in the IPG housing 122 may include control circuitry having one or more microprocessors and recharging circuitry, pulse generating circuitry, communication circuitry, and a power source for the device.

The IPG 110 is usually implanted within a subcutaneous pocket created under the skin by a physician. The leads 112 and 114 are typically mechanically and electrically coupled to the IPG 110 and thus may be used to conduct the electrical pulses from the implant site of the IPG 110 to the targeted nerve tissue via a plurality of stimulation electrodes 120. For example, the stimulation electrode region 118 of leads 112 and 114 may be positioned within the epidural space of the patient to deliver electrical stimulation to spinal nerves to treat chronic pain of the patient.

Referring now to FIG. 2, there is illustrated a block diagram of a stimulation system 200 having an implantable pulse generator (IPG) 210 and leads 212, and an external communication/charger 224. The IPG 210 includes power circuitry 214, header 216, pulse generator 218, controller 220 and communication circuitry 222. The external communication/charger 224 communicates with the communications circuitry 222 of IPG 210 via a wireless communication link and provides functionality and control mechanism to program the operating parameters of the IPG 210. The external communication/charger 224 also transmits a charging signal that is received by IPG 210 via a wireless link and is utilized to recharge rechargeable power source 214 within IPG 210. Header 216 electrically and mechanically connects leads 212 to IPG 210. Controller 220 includes one or more microprocessors and recharging circuitry. Controller 220, power circuitry 214 and pulse generator 218 function to program, control and generate electrical stimulation signals delivered to leads 212.

Referring now to FIG. 3, there is illustrated a sectional view of header 116 as similarly shown in FIG. 1. Header 116 includes a generally elongated body 117 and is preferably adapted to receive ends of lead leads 112 and 114, and to provide electrical interfacing between the leads and IPG 110. Accordingly, header 116 of the illustrated embodiment includes cavities 310 and 312, disposed in substantial alignment with electrodes 121 of a corresponding leads 112 and 114 when fully inserted, into which electrical contact assemblies 320 may be disposed. For example, spring biased contact assemblies 320 may be disposed in each of cavities 310 and 312 to provide electrical contact with corresponding electrodes 121 of leads 112 and 114 when inserted into header 116. The contact assemblies 320 are coupled to the circuitry housed within housing 122 of IPG 112 via feedthroughs (not shown). The foregoing electrical contact assemblies preferably allow insertion of a lead end through the cavities of header during installation while providing a reliable electrical contact when the leads are fully inserted into the header. It is contemplated that header 116 could include a locking mechanism to securely hold the leads therein when fully inserted. An example of a locking mechanism that can be utilized in header 116 is a set-screw assembly (see FIGS. 4 and 5). This provides a mechanical holding force to inhibit an undesired removal of the leads from the header 116.

As illustrated in FIG. 3, header 116 includes lead insertion indicators 330 and 332, each being positioned at the end of connector bores or cavities 310 and 322, respectively. Lead insertion indicators 330 and 332 are configured to be operable between at least two operating states, such that when a lead properly engages a respective lead insertion indicator, a detectable change in state of the lead insertion indicator occurs when the lead insertion indicator changes between the first state and the second state, indicating proper insertion and continued insertion of the lead into the cavity. As can be appreciated, the lead insertion indicators 330 and 332 could be mounted within header 116 using various mounting techniques.

Referring now to FIGS. 4 and 5, there is illustrated an embodiment of a lead insertion indicator 400 utilized in the present invention. In this particular embodiment, lead insertion indicator 400 includes a pressure or micro-force sensor 410 mounted a printed circuit board (“PCB”) 411, both of which are mounted to or secured to a set-screw block 412. For illustration clarity, set-screw block 412 is shown rotated so that the set-screw does not block the illustration of the other elements. The sensor 410 and PCB 411 can be mounted to set-screw block 412 utilizing a variety mounting techniques, such as, but not limited to, the use of mechanical fasteners or chemical fasteners, such as adhesives. Set-screw block 412 with PCB 411 and sensor 410 are inserted into the end of cavity of the header, whereby a connection wire (not shown), from sensor 410 is electrically connected to a feedthrough wire (not shown) for electrical connection between sensor 410 and the circuitry in the IPG.

In operation, as illustrated in FIG. 4, prior to the insertion of lead, such as by way of example lead 114 of FIGS. 1 and 3, into set-screw block 412, sensor 410 is in a first position or state. Upon the proper full insertion of lead 114 into the header and into set-screw-block 412, the end of lead 114 engages sensor 410, causing sensor to change from the first operating state to a second operating state. By way of example, if sensor 410 were a piezoresistor force sensor, in the first operating state when the lead is not in contact with sensor 410, force sensor would have a first resistive value and have a corresponding output indicative of the first operating state. When the end of lead 114 comes into contact with sensor 410 changing the sensor, the resistance of the force sensor changes when flexed under the applied force of the lead, thereby having a second resistive value and a corresponding output indicative of the second operating state. Similarly, when lead 114 is removed far enough from set-screw block 412 so as to disengage sensor 410, the force sensors changes from the second operating state back to the first operating state.

The output of sensor 410 indicating either of the operating states of sensor 410 is communicated to the IPG. The IPG can communicate information corresponding to the operating state of sensor 410 to the external communication/charger 224 such that a user can check, based upon the indication of the operating state of sensor 410, whether the lead is properly inserted into the header and engaging sensor 410. This enables a user to check the status of the proper insertion of the lead into the header prior to the implantation of the IPG into the patient, and if needed, to be able to check the continued proper insertion of the lead into the header subsequent to implantation of the IPG into the patient without any surgical procedures, such as the removal the IPG from the patient to check the status of the lead.

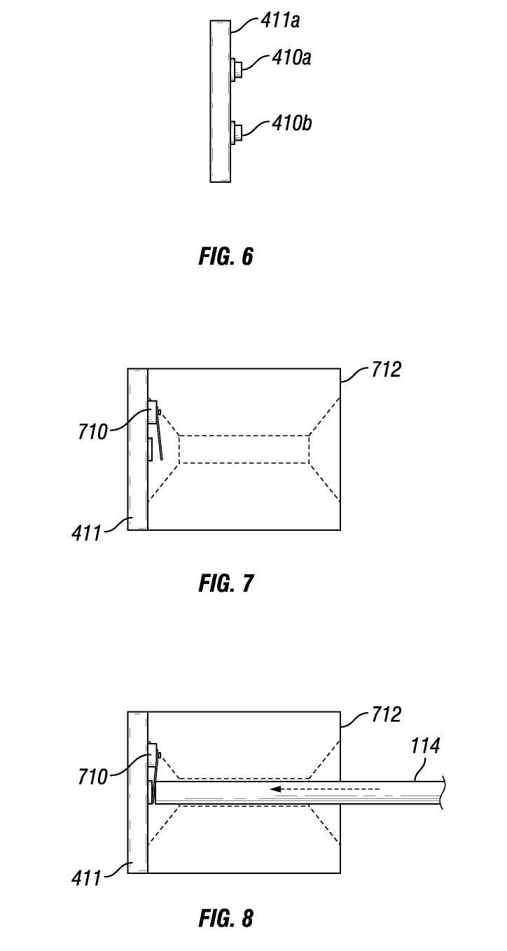

Referring now to FIG. 6, there is illustrated an alternative embodiment of the use of the micro-force sensor 410 as similarly shown in FIGS. 4 and 5. As illustrated, two micro-force sensors 410a and 410b are mounted to a single PCB 411a, which would be mounted to or secured to set-screw blocks with each of the micro-force sensors 410a and 410b in a separate set-screw block. The sensors 410a and 410b and PCB 411a can be mounted to set-screw blocks utilizing a variety mounting techniques, such as, but not limited to, the use of mechanical fasteners or chemical fasteners, such as adhesives. PCB 411a and sensors 410a and 410b, subsequent to their attachment to set-screw blocks would be inserted into the end of cavity of a header with each of the sensors 410a and 410b setup in alignment with two adjacent cavities for receiving leads, whereby a connection wire (not shown), from the sensors 410a and 410b would be electrically connected to feedthrough wires (not shown) for electrical connection between sensors 410a and 410b and the circuitry in the IPG.

In operation, as similarly described with reference to the embodiments of FIGS. 4 and 5, prior to the insertion of leads into a header, each of the sensors 410a and 410b would be in a first position or state. Upon the proper full insertion of leads into the header and into corresponding set-screw blocks, the ends of the leads would engage the sensors 410a and 410b, causing corresponding sensors to change from the first operating state to a second operating state. By way of example, if sensors 410a and 410b were piezoresistor force sensors in the first operating state when the leads were not in contact with sensors 410a and 410b, each sensor would have a first resistive value and have a corresponding output indicative of the first operating state of that sensor. When the end of leads come into contact with each of sensors 410a and 410b, the resistance of the sensors change when flexed under the applied forces of the leads, thereby having second resistive values and corresponding outputs indicative of the second operating state of each of the sensors.

The output of sensors 410a and 410b indicating either of the operating states of the sensors are communicated to the IPG. The IPG can communicate information corresponding to these operating states to the external communication/charger 224 such that a user can check, based upon the indication of the operating state of sensor 410, whether the lead is properly inserted into the header and engaging sensor 410. This enables a user to check the status of the proper insertion of the lead into the header prior to the implantation of the IPG into the patient, and if needed, to be able to check the continued proper insertion of the lead into the header subsequent to implantation of the IPG into the patient without any surgical procedures, such as the removal the IPG from the patient to check the status of the lead.

Referring now to FIGS. 7 and 8, there is illustrated another embodiment of a lead insertion indicator utilized in the present invention. In this particular embodiment, lead insertion indicator includes a lever switch 710 mounted to a printed circuit board (“PCB”) 411, both of which are mounted to mount or block 712. The switch 710 and PCB 411 can be mounted to block 712 utilizing a variety mounting techniques, such as, but not limited to, the use of mechanical fasteners or chemical fasteners, such as adhesives. Block 712 with PCB 411 and switch 710 are inserted into the end of cavity of the header, whereby a connection wire (not shown), from switch 710 is electrically connected to a feedthrough wire (not shown) for electrical connection between switch 710 and the circuitry in the IPG.

In operation, as illustrated in FIG. 7, prior to the insertion of lead, such as by way of example lead 114 of FIGS. 1 and 3, block 712, switch 710 is in an open or first position or state. Upon the proper full insertion of lead 114 into the header and into block 712, the end of lead 114 engages switch 710, causing switch 710 to change from an open state to a closed state, or from a first operating state to a second operating state.

The IPG, being electrically connected to switch 710, can communicate information corresponding to the operating state of switch 710 to the external communication/charger 224 such that a user can check, based upon the indication of the operating state of switch 710, whether the lead is properly inserted into the header and engaging switch 710. This enables a user to check the status of the proper insertion of the lead into the header prior to the implantation of the IPG into the patient, and if needed, to be able to check the continued proper insertion of the lead into the header subsequent to implantation of the IPG into the patient without any surgical procedures, such as the removal the IPG from the patient to check the status of the lead.

Although the present invention is described herein to being disposed in the header of an IPG, it is contemplated to be within the scope of the invention that the present invention could also be incorporated into other implantable devices, such as but not limited to, the connector portions of lead extenders and the like.

As will be recognized by those skilled in the art, the innovative concepts described in the present application can be modified and varied over a wide range of applications. Accordingly, the scope of patented subject matter should not be limited to any of the specific exemplary teachings discussed above, but is instead defined by the following claims.

What is claimed is:

1. A medical device, comprising:
a lead;
a housing having a communication device therein;
a header connected to the housing, the header having a cavity extending from an opening at a first end of the header towards a second end of the header, the cavity configured to receive at least a portion of the lead; and
an lead insertion indicator operable between a first operating state and a second operating state, the lead insertion indicator being disposed within the cavity of the header such that when the lead is not engaged with the lead insertion indicator, the lead insertion indicator operates in the first operating state, and when the lead engages the lead insertion indicator, the lead insertion indicator operates in the second operating state.

2. The medical device as recited in claim 1, wherein the lead insertion indicator communicates a first signal to the communication device in the housing, the first signal corresponding to the operating state of the lead insertion device when the lead insertion indicator is operating in the first operating state.

3. The medical device as recited in claim 2, wherein the lead insertion indicator communicates a second signal to the communication device in the housing, the second signal corresponding to the operating state of the lead insertion device when the lead insertion indicator is operating in the second operating state.

4. The medical device as recited in claim 3, wherein the communication device communicates with a user the operating state of the lead insertion device base upon the signal received from the lead insertion indicator.

5. The medical device as recited in claim 4, wherein the lead insertion indicator includes a switch.

6. The medical device as recited in claim 4, wherein the lead insertion indicator includes a force sensor.

7. A medical lead connector, comprising:
an elongated body having a cavity extending from an opening at a first end of the body towards a second end of the body, the cavity configured to receive at least a portion of a medical lead; and
an lead insertion indicator operable between a first operating state and a second operating state, the lead insertion indicator being disposed within the cavity of the body such that when the lead is not engaged with the lead insertion indicator, the lead insertion indicator operates in the first operating state, and when the lead engages the lead insertion indicator, the lead insertion indicator operates in the second operating state.

8. The medical lead connector as recited in claim 7, wherein the lead insertion indicator communicates a first signal to a communication device in an implantable pulse generator, the first signal corresponding to the operating state of the lead insertion device when the lead insertion indicator is operating in the first operating state.

9. The medical lead connector as recited in claim 8, wherein the lead insertion indicator communicates a second signal to the communication device in the implantable pulse generator, the second signal corresponding to the operating state of the lead insertion device when the lead insertion indicator is operating in the second operating state.

10. The medical lead connector as recited in claim 9, wherein the lead insertion indicator communicates an indication of the operating state of the lead insertion device to a user through the communication device.

11. The medical device as recited in claim 10, wherein the lead insertion indicator includes a switch.

12. The medical device as recited in claim 10, wherein the lead insertion indicator includes a force sensor.

13. A stimulation system, comprising:
an implantable pulse generator (IPG), the IPG including a header;
the header having a cavity extending from an opening at a first end of the header towards a second end of the header, the cavity configured to receive at least a portion of a lead; and
an lead insertion indicator operable between a first operating state and a second operating state, the lead insertion indicator being disposed within the cavity of the header such that when the lead is not engaged with the lead insertion indicator, the lead insertion indicator operates in the first operating state, and when the lead engages the lead insertion indicator, the lead insertion indicator operates in the second operating state.

14. The stimulation system as recited in claim 13, wherein said IPG includes a communication device, and further wherein the lead insertion indicator communicates a first signal to the communication device, the first signal corresponding to the operating state of the lead insertion device when the lead insertion indicator is operating in the first operating state.

15. The stimulation system as recited in claim 14, wherein the lead insertion indicator communicates a second signal to the communication device, the second signal corresponding to the operating state of the lead insertion device when the lead insertion indicator is operating in the second operating state.

16. The stimulation system as recited in claim 15, wherein the communication device communicates with a user the operating state of the lead insertion device base upon the signal received from the lead insertion indicator.

17. The stimulation system as recited in claim 16, wherein the lead insertion indicator includes a switch.

18. The stimulation system as recited in claim 16, wherein the lead insertion indicator includes a force sensor.
